(19) United States
(12) Patent Application Publication
(10) Pub. No.: US 2025/0381036 A1
(43) Pub. Date: Dec. 18, 2025

(54) COAPTATION DEVICE

(71) Applicants: SINGAPORE HEALTH SERVICES PTE LTD, Singapore (SG); TRISAIL MEDICAL PTE LTD, Singapore (SG)

(72) Inventors: James R. Watson, Santa Rosa, CA (US); Khung Keong YEO, Singapore (SG)

(21) Appl. No.: 18/877,672

(22) PCT Filed: Jun. 28, 2023
(86) PCT No.: PCT/US2023/069304

§ 371 Date: Dec. 20, 2024

Related U.S. Application Data
(60) Provisional application No. 63/367,198, filed on Jun. 28, 2022.
(60) Provisional application No. 63/367,197, filed on Jun. 28, 2022.

(51) Int. Cl. A61F 2/24

(52) U.S. Cl. CPC A61F 2/246; A61F 2210/0014; A61F 2220/0008; A61F 2220/0075; A61F 2230/0071; A61F 2230/0091

(57) ABSTRACT

An implantable prosthesis and delivery’ system for treating tricuspid valve regurgitation. The system is configured for pre-loading into a percutaneous delivery’ system and includes a self-expanding anchoring stent with an attached and positionable coaptation member. The stent is implanted in the inferior vena cava proximate the right atrium and is connected to the coaptation member via a multi-directional coupler and gimbal assembly. The coaptation member is fabricated from a porous or semi-porous material formed over a wire frame and is configured, possibly with leaflet matching curvature, before implantation. When deployed, the coaptation member self-aligns, self-inflates, and takes shape over several cardiac cycles to conform to the patient's tricuspid valve defects and to provide coaptation surfaces for native leaflets to reduce TVR.

### BACKGROUND OF THE INVENTION

### Technical Field

The present invention relates most generally to medical devices, and more particularly a tricuspid valve prosthesis that provides a high efficiency coaptation surface for use in the treatment of tricuspid regurgitation (TR) in diverse patient anatomies.

### Background Discussion

This narrative and its accompanying drawings describe a novel tricuspid valve prosthesis, highlighting novel features related to its ability to provide a high efficiency coaptation surface in the treatment of tricuspid regurgitation (TR) in diverse patient anatomies.

The tricuspid valve (TV) comprises multiple arrangements of native tissue leaflets and a corresponding circumferential tissue ring (annulus) within the right heart structure. The inferior vena cava (IVC) returns de-oxygenated blood to the fight atrium (RA) for subsequent flow through the TV into the right ventricle (RV) and eventually to the lungs for reoxygenation. In TVR, the tricuspid valve between the right atrium and the right ventricle, does not close properly after blood is pumped from the right atrium into the right ventricle. Improper coaptation between the native leaflets (anterior, posterior, and septal) may result from several causes, including enlargement of the TV annulus, structural damage to the chordae tendineae, papillary muscle compromise, and so forth. As a result of the improper coaptation, at high ventricular contraction pressures during ventricular systole, blood flows back from the right ventricle into the right atrium.

Designing medical devices that effectively reduce TR in patients is a challenging problem. When pharmacological interventions such as diuretics or vasodilators are ineffective, there remain two primary solutions at present: (1) a mechanical solution to remodel the TV annulus shape and size to force the leaflets closer together for coaptation, which increases the risk to fragile tissue; and (2) the insertion of a device that “closes the gap” to prevent or reduce TR in the TV via coaptation with the native leaflets.

The designs presented in this application are directed to the latter type of solution, wherein a novel and improved coaptation device is safely anchored in the IVC and easily positioned within the TV leaflets. The inventive coaptation device is unique in its freedom of movement in multiple axes to allow unencumbered contact with the native leaflets, and thus to avoid giving rise to new TR. The novel 3D shape of the coaptation member conforms to precisely “what is needed” to reduce TR.

### DISCLOSURE OF INVENTION

In its most essential aspect, the tricuspid valve prosthesis of the present invention includes an IVC stent fabricated from nickel titanium (nitinol) and configured for positioning in the IVC near the juncture of the right atrium (RA) and the IVC. The stent thereby provides anchoring for the tricuspid valve prosthesis in the IVC itself. A coupler connects the IVC stent to a gimbal, which, in turn, connects the coupler to a coaptation member (hereinafter referred to as a “coaptation sail”) and provides multi-axial rotation of the coaptation sail relative to the coupler within the TV annulus. The coaptation sail includes 3D-shaped nitinol wire frames covered or enclosed by or within various porous and non-porous fabric materials. Sutures attach the coaptation sail to the gimbal, and in embodiments the nitinol wire frame is also captured within the gimbal structure. When deployed, the fabric covered coaptation sail partly extends centrally into the TV to provide a coaptation surface for the native TV leaflets.

Delivery, implantation, and deployment of the coaptation sail is accomplished using the delivery system described in co-pending International Patent Application Serial Number PCT/US23/69296, filed Jun. 28, 2023, which application is incorporated in its entirety by reference herein.

The TR patient population includes numerous anatomical variations departing from the basic dimensions, such as IVC diameter and TV annulus size. The orientation of the patient IVC ostium (IVC ostial plane) and the distance to the TV annulus, as well as the TV annulus orientation (TV annulus plane) present additional challenges in positioning the coaptation sail, yet the orientation and position of the coaptation sail in the 3D volume of the RA and TV annulus is crucial to successful TR reduction. As such, several prosthesis capabilities are needed, and are provided, to enable the coaptation system of the present invention to treat the wide variety of TR patient population anatomies. Thus, variations may be included in embodiments of the invention without departing from the spirit and scope of the inventive concept.

### BRIEF DESCRIPTION OF THE DRAWINGS

The invention will be better understood and its various objects and advantages, other than those set forth above, will become apparent when consideration is given to the following detailed description thereof. Such description makes reference to the annexed drawings wherein:

FIG. 1 is an isometric side view of the Tricuspid valve prosthesis, not showing tensioner and anchor components;

FIG. 2A is a perspective view illustrating the gimbal and coupler assembly used in the prevent invention;

FIG. 2B is a top view thereof;

FIG. 2C is a cross-sectional side view in elevation thereof taken along section lines 2C-2C of FIGS. 2A-2B;

FIG. 2D is a highly schematic view featuring a configuration of the gimbal that enables degrees of rotation, pivoting, and swivel in relation to the coupler;

FIGS. 3A-3D are highly schematic views showing methods to enable a coaptation sail to be sheathed in a delivery system sheath while being able to self-orient into position upon unsheathing, wherein FIG. 3A shows the coaptation device assembly poised for sheathing in a delivery sheath; FIG. 3B shows the angled configuration of the coupler distal portion and gimbal in relation to the coupler proximal portion after unsheathing; FIG. 3C(a) schematically again shows the sheathed alignment; and FIG. 3C(b) shows the angled alignment after unsheathing;

FIG. 4A is a cross-sectional side view in elevation showing the gimbal and coupler assembly sheathed in a delivery system sheath;

FIG. 4B is an isometric view of the gimbal and coupler assembly unsheathed before pivoting of the gimbal relative to the coupler;

FIG. 4C is a cross-sectional side view showing the gimbal and coupler unsheathed and in an angled configuration;

FIGS. 5A-5C are schematic cross-sectional side views of TV leaflets showing possible coaptation configurations or scenarios, wherein FIG. 5A shows full coaptation of the leaflets and no coaptation gap, FIG. 5B shows a wide coaptation gap in the TV leaflets, and FIG. 5C shows a narrow coaptation gap;

FIGS. 6A-6C are schematic side views in elevation illustrating how the coaptation sail changes shape over several cardiac cycles to create a coaptation surface that follows the native leaflets during the cardiac cycle, wherein FIG. 6A shows the coaptation sail shape when first deployed, FIG. 6B shows the shape at an early stage of blood filling and coagulation, and FIG. 6C shows the coaptation final sail shape when it is configured in vivo to effectively fill the coaptation gap and prevent TR;

FIGS. 7A-7F are highly schematic views illustrating various coaptation sail configurations that enable a sail to be sheathed in a delivery system sheath yet self-orient and self-align in position upon unsheathing, wherein FIGS. 7A and 7B are front and end views showing a wedge shaped coaptation sail; FIG. 7C is a top view showing a pre-curved configuration; FIG. 7D is a top view showing a lanceolate shape; FIG. 7E is a front view showing a heart shape with an inferior bifurcation; and 7F is a front view of a heart shaped coaptation sail;

FIGS. 8A-8C [prev. 6B] are highly schematic views showing still further coaptation sail configurations adapted for use in curved coaptation commissures, wherein FIG. 8A is an end view showing a curved coaptation sail configured for a curved coaptation commissure; FIG. 8B is front view showing a bifurcated or pleated coaptation sail, also adapted for improved coaptation around a curve; and FIG. 8C is a top view thereof,

FIG. 9 is a schematic view showing the degrees of freedom and the axes along which the coaptation sail can be translated and rotated, respectively, so that the operator (physician) can effect precise positioning of the coaptation sail within the TV annulus;

FIG. 10 is a perspective view of an alternative embodiment of the tricuspid valve prosthesis, here seen with steering and tensioning rod subassemblies connected for precisely controlling the placement, positioning, implantation, and deployment of the coaptation device of the present invention;

FIGS. 11A-11C are perspective views of select components of the steering subassembly shown with the coaptation device, here showing the distal end of the steering tube, gimbal, and coupler assembly, including the nitinol wires of the coaptation sail, with FIG. 11A being an upper front view, FIG. 11B being an exploded view thereof, and FIG. 11C being a side view showing the components assembled and operatively connected;

FIGS. 12A-12C are perspective views showing several orientations of one steering system at various flexure amounts and rotations, thereby highlighting its degrees of freedom and ranges of motion for positioning the placing the coaptation sail in the TV, wherein FIG. 12A shows the steering tube flexing away from the side of the IVC stent to which it is attached, FIG. 12B show it flexing toward the attachment side, and FIG. 12C shows the steering tube flexing significantly more under operator control;

FIG. 13 is a detailed view of the stent and steering system subassembly of the inventive delivery system and a proximal portion of the coaptation sail, here featuring the connection between the stent and the steering tube subassembly and the distal end of the steering tube coupling to the coupler/gimbal assembly and ultimately the coaptation sail;

FIG. 14 is an upper perspective view showing the components comprising the steering system and tensioning rod subassemblies;

FIGS. 15A-15C are perspective views of the tensioning rod assembly shown in FIG. 14, FIG. 15, and FIG. 17, FIG. 18a being an assembly view, FIG. 18B being an exploded view, and FIG. 18C being a cross-sectional view thereof;

FIG. 16 is detailed exploded view of select components of the tensioning rod subassembly shown in FIGS. 15A-15C;

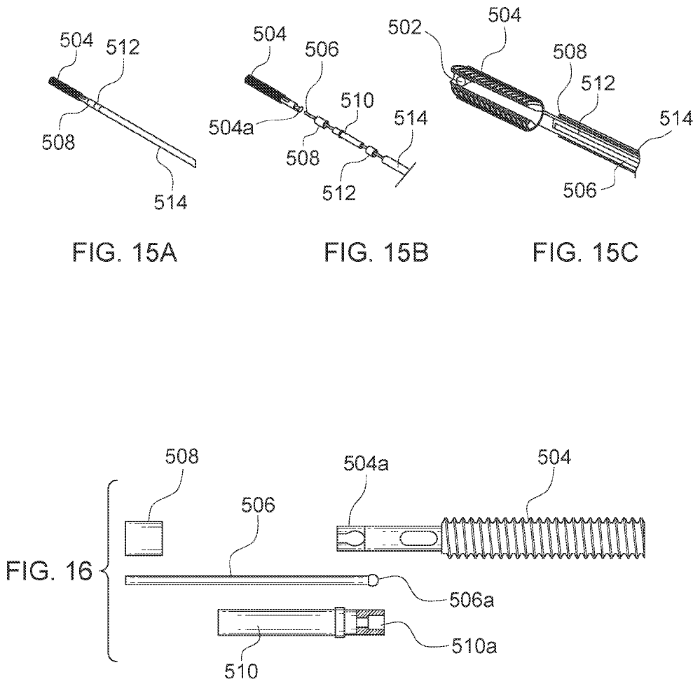

FIG. 15 is an upper perspective view showing the tensioning rod subassembly coupled to the steering system subassembly, which is, in turn connected to the stent;

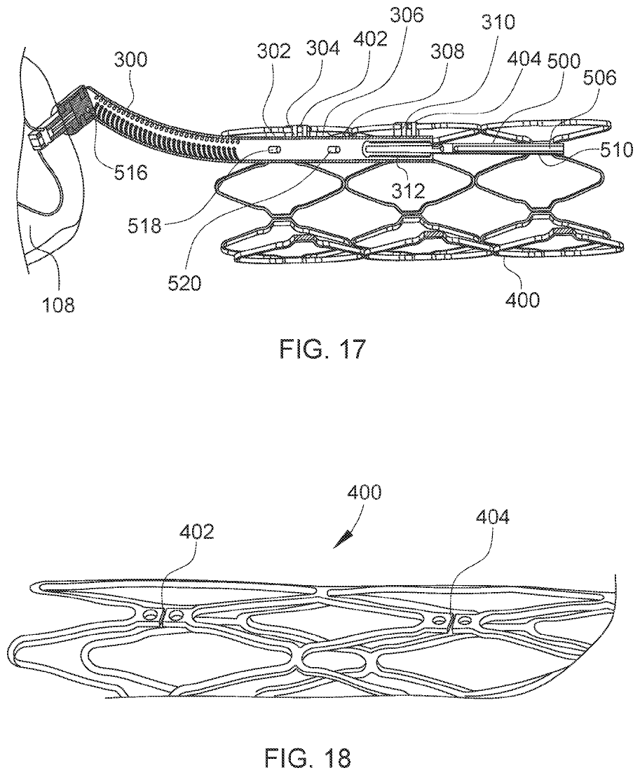

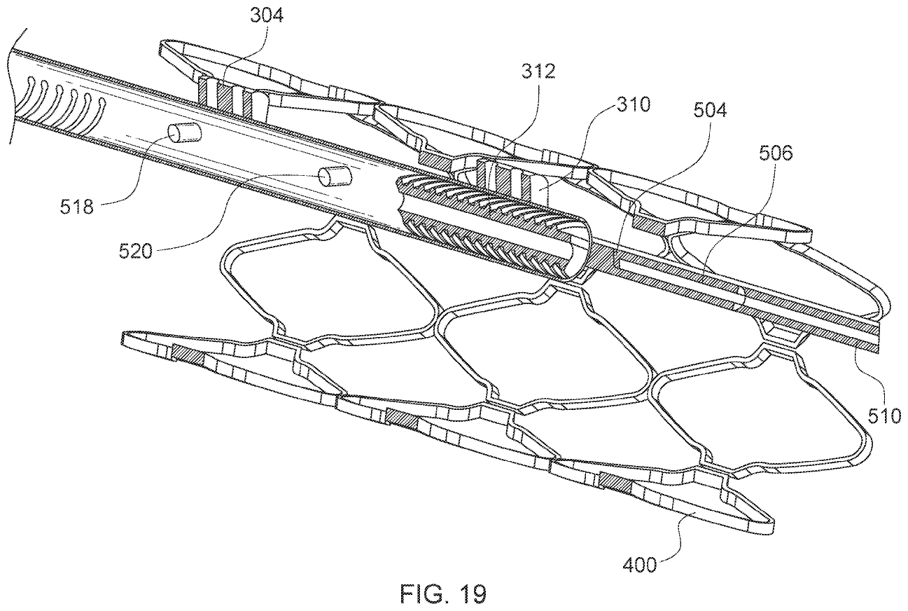

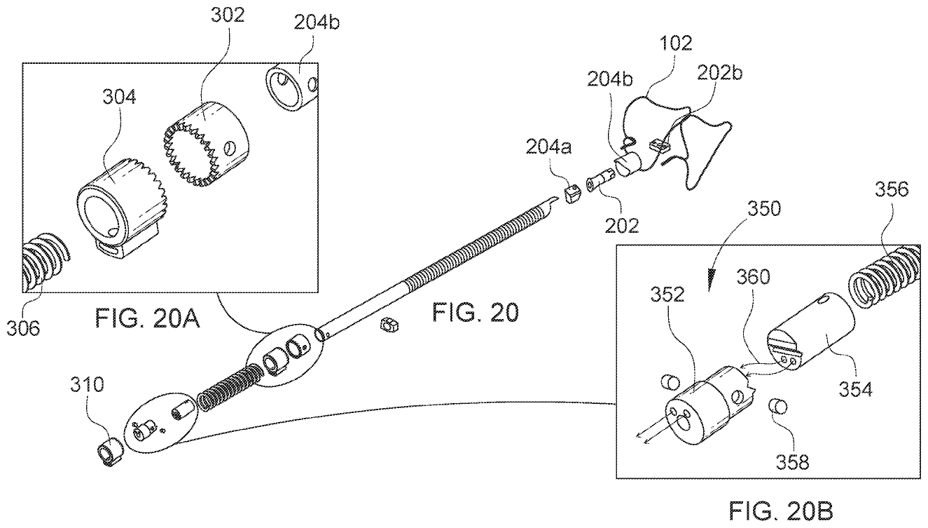

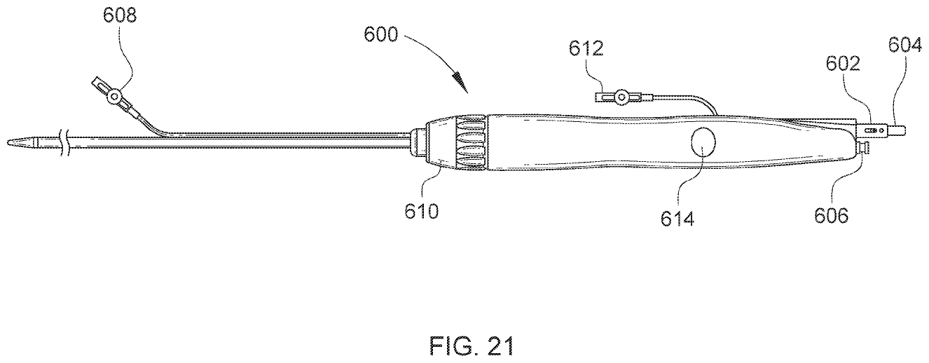

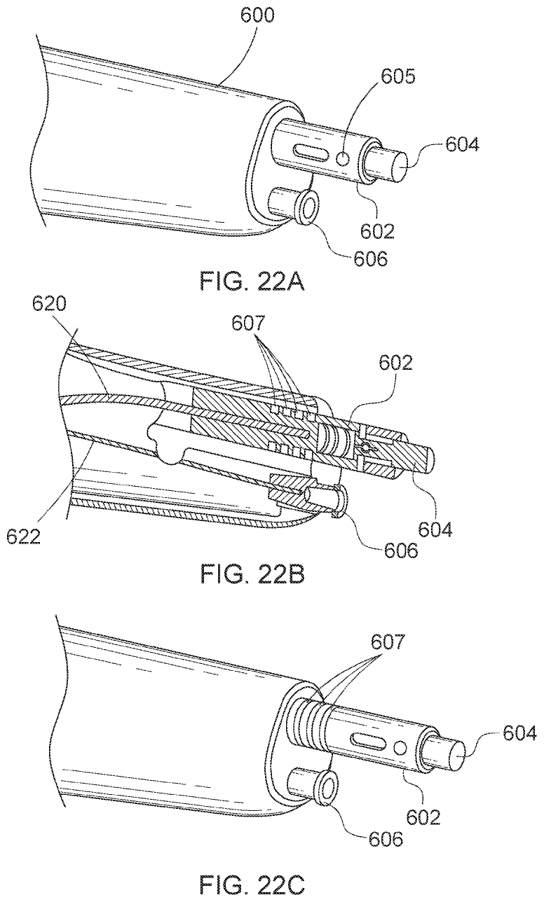

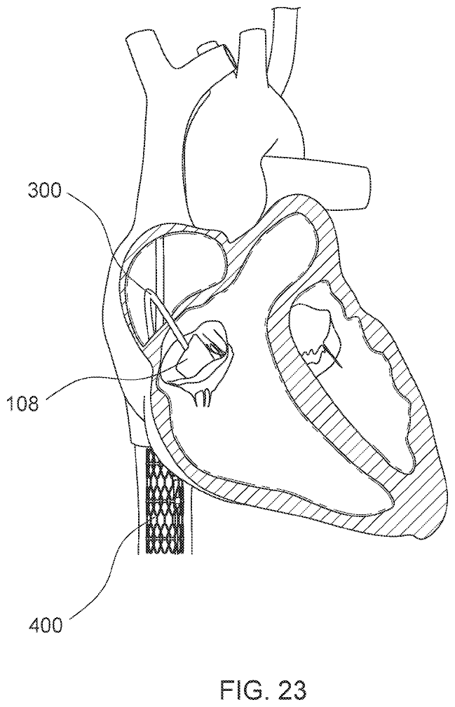

FIG. 16 is a detailed view of attachment structure for connecting the steering tube to the stent;

FIG. 17 is a detailed cross-sectional view of the steering system and tensioning rod subassemblies taken along section lines 17-17 of FIG. 13;

FIG. 18 is a detailed perspective view of attachment structure for connecting the steering tube to the stent;

FIG. 19 is a more detailed view of the steering tube and tensioning rod subassemblies shown in FIG. 17;

FIGS. 20A-20C are perspective views showing details of an alternative tensioning member locking mechanism, with FIG. 20A being an exploded view, FIG. 20B being a detailed view showing details from window 20B of FIG. 20A, and FIG. 20C showing details, with detailed views incorporated, showing an alternative tensioning member locking mechanism;

FIG. 21 is a front view in elevation of the control handle for the prosthesis delivery system; and

FIGS. 22A-22C are perspective views of the proximal end of the delivery system control handle, FIG. 22A being an assembly view, FIG. 22B being a cross-sectional view, and FIG. 22C being an assembly view showing the tension knob extended.

### BEST MODE FOR CARRYING OUT THE INVENTION

The following structures, features, and functions enable the inventive tricuspid valve prosthesis and its delivery system to treat larger and diverse TR patient anatomies. Each provides advantages either individually or in combination. The new prosthesis elements include a self-aligning gimbal, a self-filling 3D coaptation sail, a coupler, a pre-curved prosthesis configuration (that does not utilize a tensioner system), and an auto-rotation mechanism for the coaptation sail.

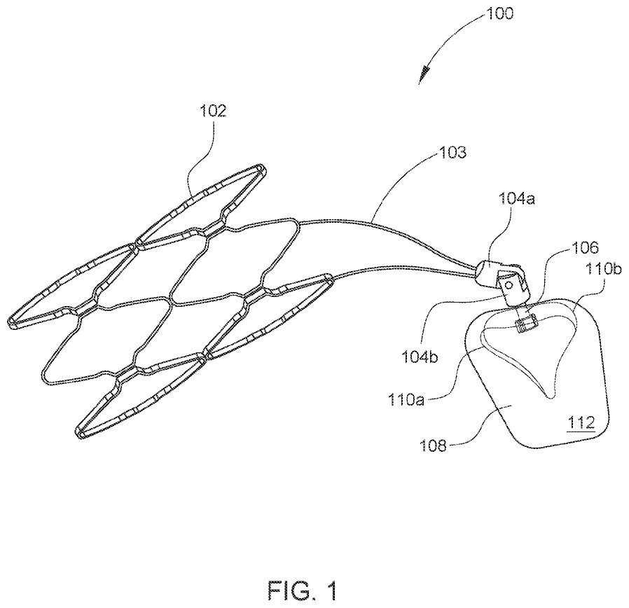

Referring first to FIG. 1, there is shown in a perspective view the tricuspid valve prosthesis of the present invention 100. This view does not feature either tensioner or anchor components, which are assumed. A nitinol IVC stent 102 is positioned in the IVC near the juncture of the right atrium (RA) and the IVC. The stent provides anchoring for the tricuspid valve prosthesis at deployment and is effected in the IVC itself. A coupler 104 includes a proximal portion 104a which is tethered to the IVC stent 102 via a nitinol or other medical grade wire 103, a distal portion 104b that connects to a gimbal 106. The gimbal is then connected to a coaptation sail 108.

The coupler/gimbal assembly provides multi-axial rotation of the coaptation sail relative to the coupler within the TV annulus. The coaptation sail itself comprises internal 3D-shaped nitinol wire frames 110a, 110b, covered with porous or semi-porous material, such as a woven 112. The material may be selected from any of a number of porous, semi-porous, and even non-porous materials, such as a woven fabric, a polymer barrier, polyurethane foam (PU), reticulated polyurethane, polytetrafluoroethylene (PTFE), etc. Polyester sutures are employed to attach the coaptation sail to the gimbal. Upon deployment, the coaptation sail will extend, at least partly, and generally centrally into the TV. This provides a coaptation surface for the native TV leaflets sufficient to resolve the valve coaptation gaps.

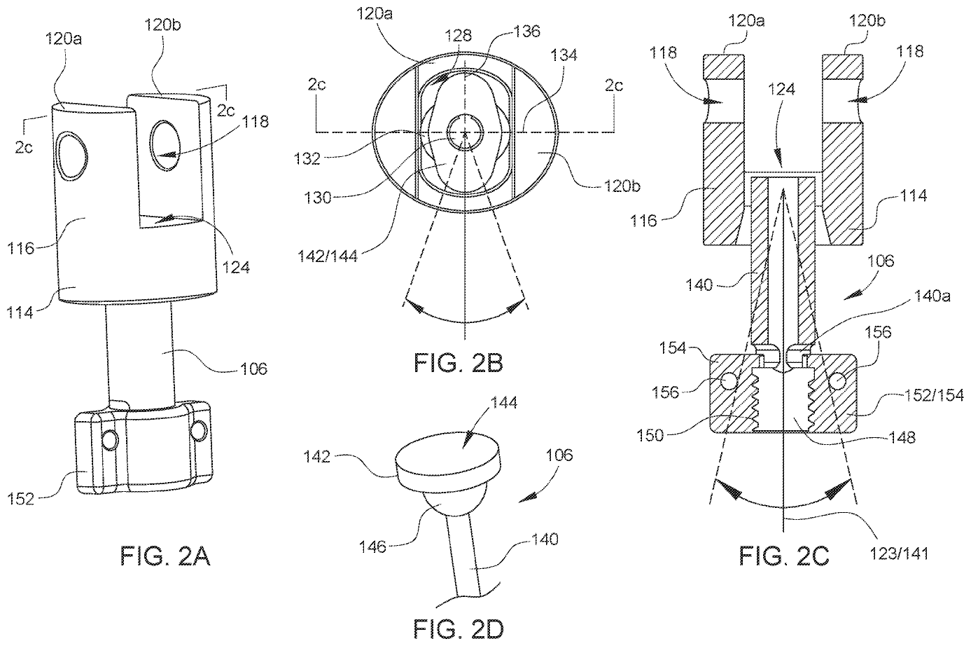

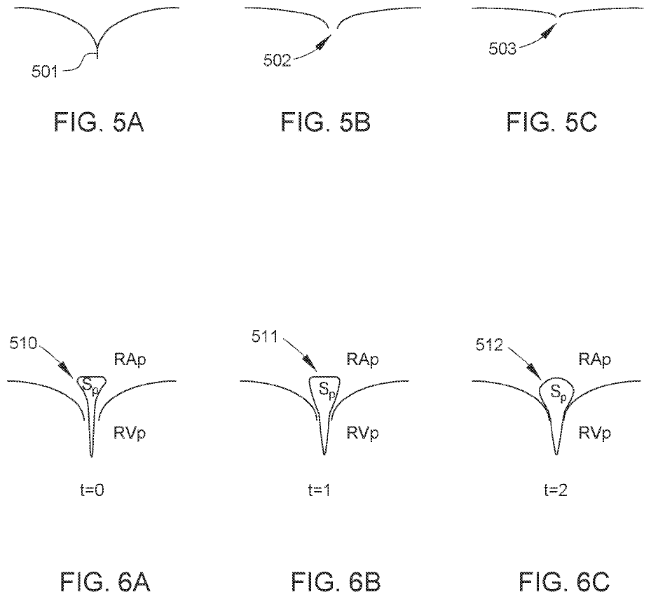

The isometric view of FIG. 2 shows that the distal portion 104b of the coupler 104 includes a base 114 and an integral yoke 116 having axially aligned through holes 118 through arms 120a, 120b for passing an axle or pin (see FIGS. 5A-5C) to connect the distal portion 104b to the proximal portion 104a of the coupler 104. The coupler base 114 includes a cylindrical frustum passage 122 centrally and longitudinally disposed between arms 120a, 120b. The platform 124 between the base of the arms includes a first oval well or recess 126 with a sidewall 128 and has a center 130 over aligned with the center axis of the cylindrical frustum passage. A deeper, second oval recess 132 also has a center over the center axis 123 of the cylindrical frustum passage and includes a major axis 134 between its vertices normal to the major axis 136 of the first oval recess.

The gimbal 106 includes a cylindrical shaft 140 having a central axis 141, which is coincident with the central axis 123 of the cylindrical frustum passage 122 when the shaft inserted through the cylindrical frustum passage 122 in the coupler distal portion base 114, wherein the clearances between the shaft and the cylindrical frustum passage are such that the shaft may pivot across the cylindrical frustum passage axis in an approximate 20-50 degree arc (see FIG. 2C). In embodiments, the pivot may be a substantially symmetrical swing, but it need not be, and in some embodiments, the swing may be tailored to a particular patient and made with an asymmetrical range of motion. Gimbal shaft movement to support the self-aligning function of the coaptation sail is further enabled by the gimbal head 142, which is seated in the first oval recess 126, and has a generally planar ovoid top 144 top and, in embodiments, may include a hemispherical ball 146 disposed between the shaft 140 and the top 144. The vertices of the ovoid head have clearances from the sidewalls of the first ovoid recess such that the head may also rotate within the cylindrical frustum passage approximately 10-40 degrees. Summarily, it will be appreciated that with respect to the coupler, the gimbal both pivots and rotates.

The distal end 148 of the gimbal shaft 140 includes male threads 150 onto which a gimbal wing nut 152 is threadably attached, securing the nitinol wire ends within the gimbal. More specifically, in embodiments the wires of the nitinol frame pass through a slot or hole 140a in the gimbal shaft and wrap around a circumferential channel, in which they are captured by the gimbal nut when threadably installed on the gimbal shaft. In embodiments, sutures may be employed to secure the material covering, embedding, or enclosing the nitinol wire frame. The wings 154 of the gimbal wing nut include holes 156 further facilitating attachment (via sutures) of the coaptation sail fabric to the gimbal assembly.

So configured, and as seen in FIGS. 2B and 2C, the gimbal shaft rotates within the coupler cylindrical frustum passage and the head rotates within the recesses in the coupler platform.

The inventive coaptation device (and more specifically the configuration and components of the coaptation sail itself) provides a three-dimensional surface for the native leaflets to contact (coaptate) so that blood does not flow into the RA during RV contraction. Previous devices having generally planar configurations and coaptation surfaces may have effectively extended the native leaflet, but in many instances the devices were inadequate to resolve the coaptation gap and thus inadequate to reduce TR.

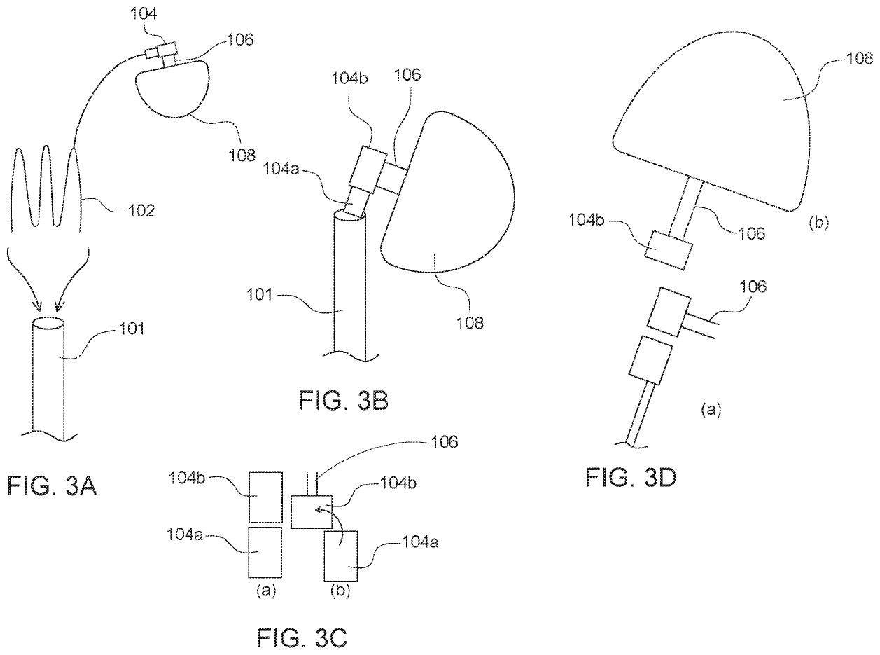

FIGS. 3A-3E are highly schematic views showing the contact regions of the coupler 104 and gimbal 106 upon retraction of the system into a delivery sheath 101. Because the coupler distal portion 104b rotates about an axle or pin 119 disposed through holes 118 in the yoke 116 connecting the coupler distal portion with the coupler proximal portion 104a, the gimbal shaft is brought into axial alignment with the axis of the delivery sheath 101 when the components are pulled into the sheath for delivery (FIGS. 3A, 3C(a), and 3D(a)). When unsheathed during delivery, the distal portion 104b of the coupler rotates into an angled configuration, possibly aided by a pusher bar operated by the physician (FIGS. 3B, 3C(b), and 3D(b)).

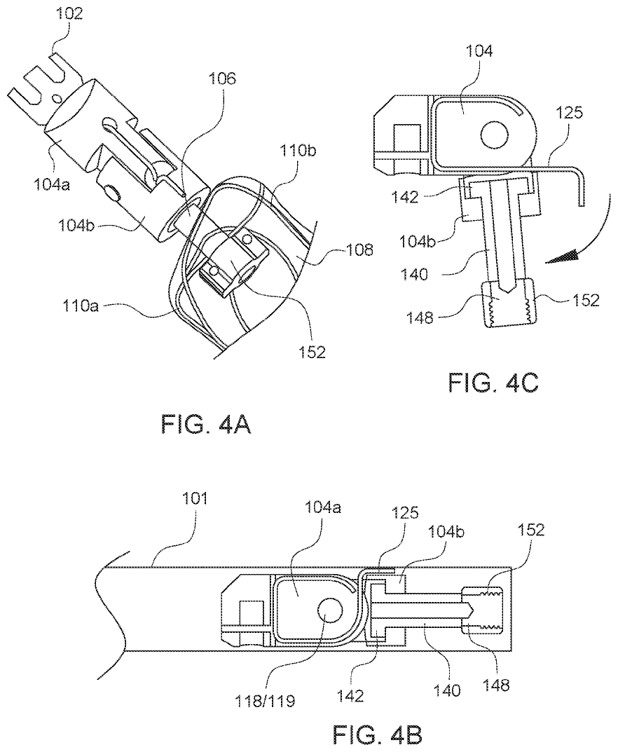

This operation is further illustrated in FIGS. 4A-4C, where it can be seen that when urged into a delivery sheath (FIG. 4A), in embodiments a nitinol wire torsion spring 125 is captured within the cylindrical sheath walls and moved into a bent configuration, thereby placed into tension. When unsheathed, the spring 125 straightens and imparts an angular force on the distal portion 104b to rotate it around axle 119 and into an angled relationship to the proximal portion 104a, such that when delivered, the axis 141 of the gimbal tilts laterally and down to be generally coaxial with the convergence of the commissures of the septal, posterior and anterior leaflet coaptation lines. This auto-rotation mechanism of the coupler/gimbal/sail assembly helps to achieve the optimal orientation of the coaptation sail, wherein the top of the coaptation sail is preferably parallel to the TV annulus. This will help to ensure maximal coaptation of the coaptation sail with the native leaflets.

Looking next to FIGS. 5A-5C, several schematic views show coaptation of TV leaflets FIG. 5A 501, and coaptation gaps, wide 502, and narrow 503, respectively, the latter shown in FIGS. 5B, 5C.

In embodiments, the nitinol wires of the coaptation sail may be pre-curved to better match the target TV. The stent may also be configured with a pre-curved section but without a tensioner and anchor system. Such an alternate configuration may be desired to reduce procedural complexity. It will be appreciated that multiple pre-curved variations may be available for the physician to address a specific patient anatomy.

FIGS. 6A-6C show performance of the coaptation sail upon deployment to fit the several coaptation scenarios 510, 511, 512, and the kinds of gaps shown in FIGS. 5B-5C. For simplicity, corresponding chordae and papillary muscles are not shown. And for the purposes of this disclosure, the coaptation gaps need not be characterized, as it will be understood that the gaps may extend entirely or only partly across the anterior-septal, anterior-posterior, or septal-posterior coaptation lines. Both narrow and wide coaptation gaps, whether partially or fully extending across the coaptation lines, will cause TR. Importantly, these views also illustrate the changing configuration of the coaptation sail over several cardiac cycles to adapt and conform to the coaptation gap and thereby create a coaptation surface that follows the native leaflets during the cardiac cycle. FIG. 6A shows the coaptation sail shape when first deployed, FIG. 6B shows the shape at an early stage of blood filling and coagulation, and FIG. 6C shows the coaptation final sail shape when it is configured after several cardiac cycles to effectively fill the coaptation gap and prevent TR.

In embodiments, the coaptation device includes the feature that when deployed, the coaptation sail may be shaped three dimensionally through the use of a semi-porous or porous material, such as, for example, fabric, woven fabric, polymer barrier, polyurethane foam (PU), reticulated polyurethane, polytetrafluoroethylene (PTFE), etc. The coaptation sail material allows blood to fill the 3D coaptation sail interior while taking the shape of the native leaflets and any gap between the leaflets without the need to use a balloon. This also provides an internal expansive structure that a physician can load into a delivery system sheath.

The views in FIGS. 6A-6C illustrate how the coaptation sail changes shape over time to create a coaptation surface that follows the native leaflets. The 3D coaptation sail's blood-filling feature occurs naturally during the cardiac cycle due to the difference in pressures in the RA and RV respectively. Initially the pressure within the coaptation sail (Sp) equals the right atrium pressure (RAp), since it is deployed within the right atrium (RA).

At t=0, 510, as the coaptation sail crosses the TV annulus and is positioned between the native leaflets, it is subjected to a pressure differential, since RVp>RAp during systole. During diastole the pressures are more equal and have minimal effect on the coaptation sail pressure. Note that the pressure difference permeates the coaptation sail's structure during the cardiac cycle.

At t=1, 511, the porous coaptation sail material allows non-coagulated blood and other blood constituents to enter the coaptation sail's interior, since blood flows from high to low pressure, and to slowly fill the interior. Note that as the coaptation sail is filling, it is also increasingly closing the coaptation gap, which in turn increases the pressure differential, thereby filling the coaptation sail even more fully.

In embodiments, a variably porous material construct could be used to regulate the pressure change and the resultant blood volume, as well as the coaptation sail shape. Various methods are available to control the final coaptation sail shape and prevent it from becoming a ball that unproductively floats on top of the leaflets, such as having the porosity change relative to the depth of insertion.

At t=2, 512, the coaptation sail's interior is filled with blood, and the blood is coagulating inside the coaptation sail because it is out of the turbulent blood flow. As the blood coagulates, the congealed blood is unable to pass back through the coaptation sail's porous material even though the pressure differential remains, effectively creating a 3D coaptation sail shape that matches the native leaflet's shape.

Ultimately, endothelialization of the coaptation sail's surface seals the congealed blood within the coaptation sail although it may change shape over time in response to a changing annulus.

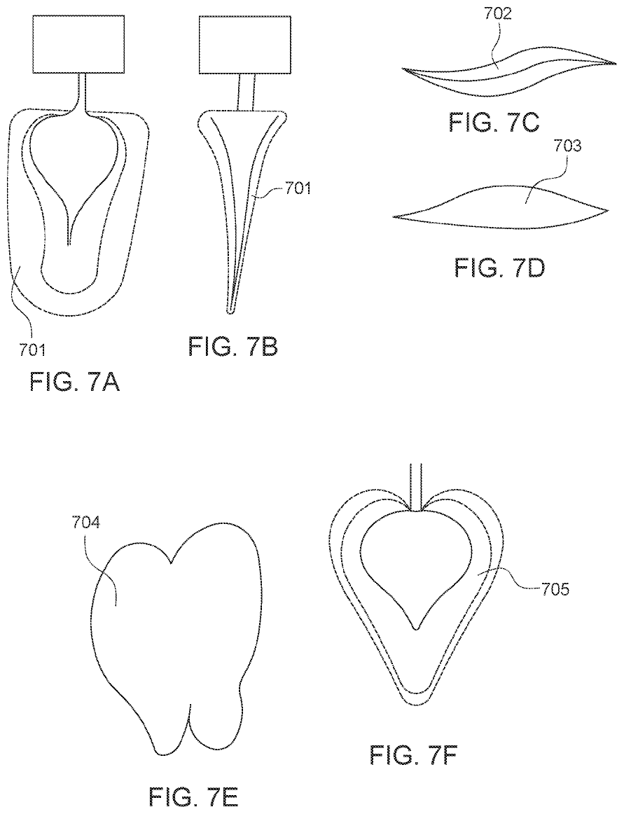

Note that the coaptation sail's coaptation range is determined by the degree of TR present and the coaptation gap size. Because the coaptation sail cross-section is generally linear at the bottom and slowly transitions to a 3D ovoid shape at the top, the amount inserted across the annulus provides the ability to treat a wide range of anatomically different TR types. Hence, the bottom planar portion of the coaptation sail effectively provides native leaflet extension while the upper portion provides TR gap filling. Hence the coaptation sail's long axis may be at various angles (not necessarily perpedicular) to the TV annulus plane based upon the patient's anatomy and the insertion depth, and it will nonetheless still achieve TR reduction. Various configurations of the coaptation sail, 701-705, are shown in FIGS. 7A-7F.

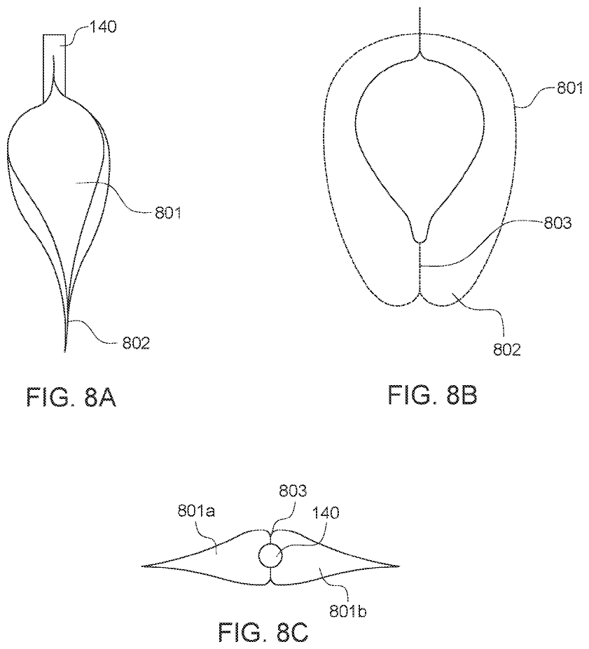

Referring to FIGS. 8A-8C, a coaptation sail 801 may include a lower planar section 802 may include one or more pleats or bifurcations 803 creating discrete lobe portions 801a, 801b to enable promote coaptation sail flexure around curved coaptation commissures to enable it to provide improved coaptation around a curve. These are shown in FIGS. 8A-8C.

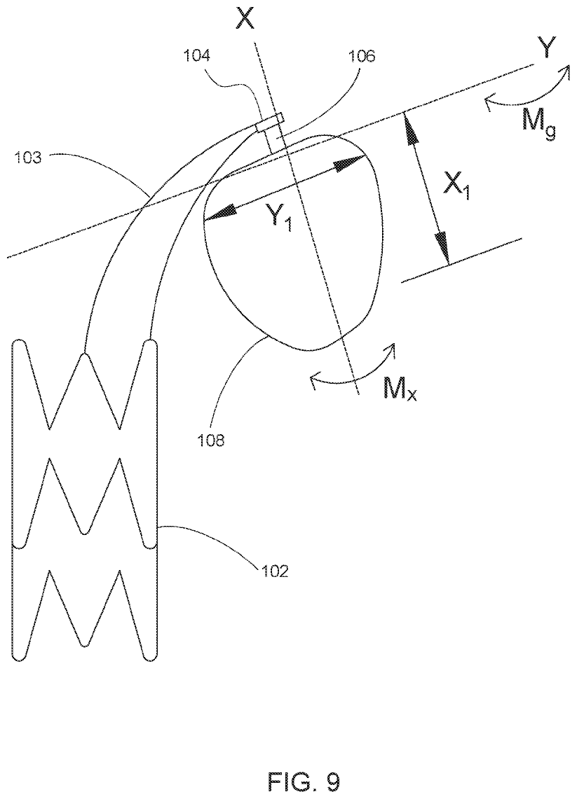

FIG. 9 is a schematic view showing the degrees of freedom indicating the ability of the physician to control the positioning of the coaptation sail within the TV annulus via delivery system handle movements and show the gimbal's relative motion to promote improved coaptation.

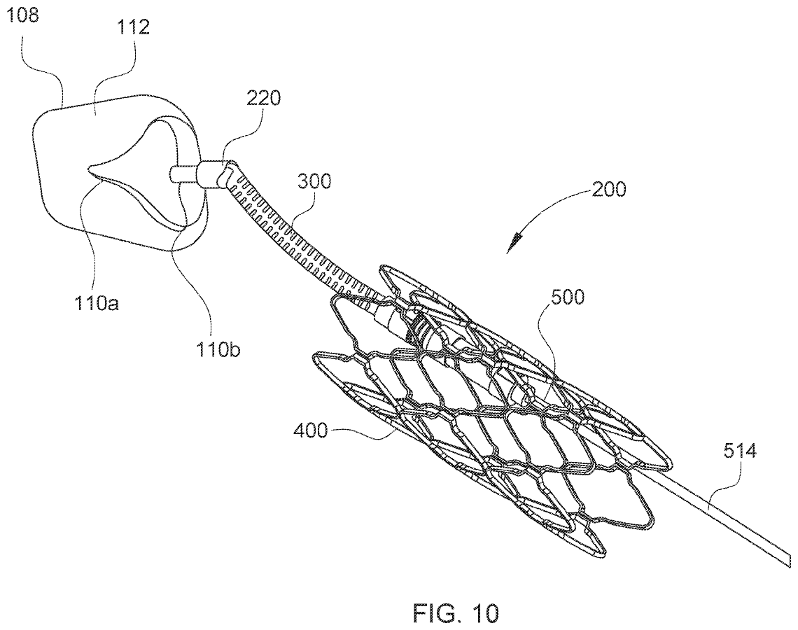

Referring next to FIG. 10, in an alternative embodiment of the inventive coaptation device 200, the device includes and is structurally and operatively connected to a delivery system that facilitates precise positioning and placement of the coaptation device during installation and deployment. The following narrative includes a description of the TV prosthesis itself, here again including a coaptation sail 108, positioned and placed by a dedicated coaptation device delivery system. The coaptation sail 108, its porous/semi-porous material cover 112, and its nitinol frame 110a, 110b, remain substantially identical to the earlier described embodiment, and thus maintain the same reference numbers. Other elements, such as the coupler and gimbal, are substantially the same but are modified for use in the novel delivery and placement system, and therefore, along with newly described elements and features, bear new numbers.

Referring next, then, to FIG. 10, it will be seen that in the alternative embodiment 200 the coaptation sail 108 includes internal 3D-shaped nitinol wires 110a, 110b, at least partly enclosed and covered in various porous and non-porous materials 112. The coaptation sail includes a proximal medial section 106 between the nitinol wires. The coaptation sail 100 extends somewhat centrally into the TV to provide a coaptation surface for the native TV leaflets.

At the proximal medial section of the coaptation sail, an alternative embodiment of the gimbal/coupler subassembly 220 connects the coaptation sail 108 to a nitinol steering tube subassembly 300. The gimbal 222 connects the coupler 224 to the coaptation sail 108 and provides multi-axial rotation of the coaptation sail, relative to the coupler 224, within the TV annulus. The coupler 224 includes a proximal portion 224a and a distal portion 224b, which capture the gimbal in a way such that the gimbal includes degrees of freedom via rotational and swivel motions relative to the coupler. The coupler 224 is connected to the distal end 300a of the steering tube with pins 300b. And the nitinol wire frame 102 of the coaptation sail 108 is connected to the distal end of the gimbal shaft 222a gimbal with a coupling clamp 222b.

The steering tube 300 and associated tensioner rod subassembly 500 is attached to the stent 400 and the delivery system handle to provide multi-axial adjustable positioning of the coaptation sail 108. Upon tensioning and/or rotation of the steering tube subassembly within the stent 400, the coaptation sail is positioned and aligned with the TV annulus engaging the native TV leaflets to treat a wide variety of anatomies. The IVC stent 400 is constructed from nitinol and is positioned in the IVC, near the juncture of the right atrium (RA) and IVC, and it provides anchoring of the tricuspid valve prosthesis in the IVC. Note that the anti-thrombogenic covering on the steering tube 300 is not shown.

The TR patient population includes many anatomical variations beyond the basic dimensions such as IVC diameter and TV annulus size. The orientation of the IVC ostium (IVC ostial plane), and the distance to TV annulus as well as the TV annulus orientation (TV annulus plane), introduce additional challenges in positioning the coaptation sail. Yet, the orientation and position of the coaptation sail in the 3D volume of the RA and TV annulus is crucial to successful TR reduction. As such, additional prosthesis and delivery system capabilities are required to ensure the inventive coaptation prosthesis and delivery system is able to treat the wide variety of TR patient population anatomies.

The following novel system provides the needed capabilities, with features and functions that enable the inventive coaptation prosthesis and delivery system to treat more diverse TR patient anatomies. Each of the capabilities provides advantages either individually or in combination. Notably, several novel aspects of the coaptation sail 100 and gimbal 222 and coupler 224 are disclosed co-pending International Patent Application, which shares the inventors of the present invention and is filed concurrently herewith, said application entitled “Coaptation Device”, incorporated in its entirety herein by reference.

The prosthesis and delivery system elements included in this disclosure include as principal components: (1) a novel gimbal design fabricated from medically suitable materials, such as polyether ether ketone (PEEK), stainless steel, titanium, etc., (2) a steering tube (with multi-axis adjustability, and fabricated from materials such as nitinol, PEEK, etc.; (3) a stent design for steering tube attachment, also fabricated from the same materials; and (4) a tensioning rod subassembly, fabricated from PEEK, stainless steel, titanium, polyimide, and etc.

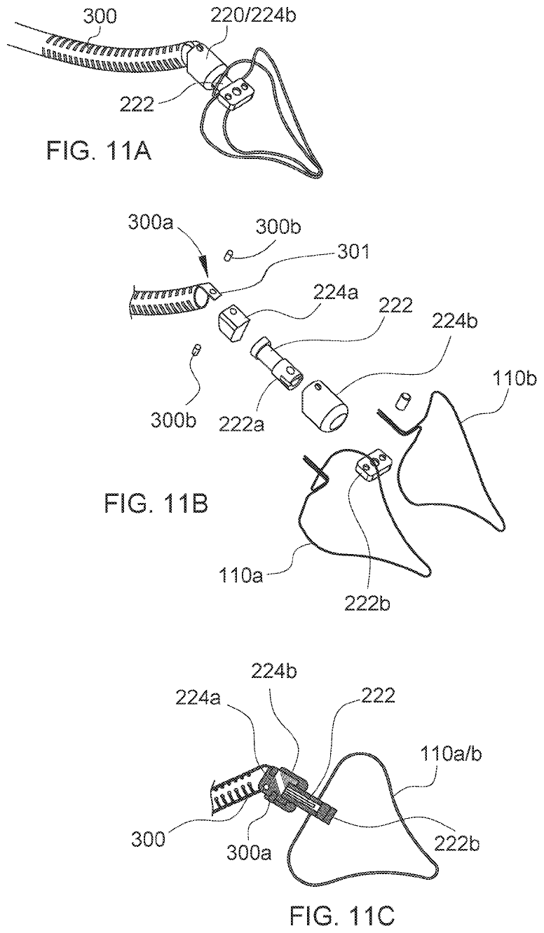

Gimbal 222 connects the coupler 224 to the coaptation sail 108 and enables multi-axial movement of the coaptation sail relative to the coupler. The ability of the coaptation sail to self-orient within the TV annulus ensures that it does not impinge on the native leaflets, causing more TR, but instead self-aligns with the coaptation commissures to increase native leaflet coaptation. Several views of the gimbal and coupler assembly are shown in FIGS. 11A-11C to illustrate the design elements. The porous and non-porous covering materials including a middle section between the nitinol wires or the porous and non-porous covering between the outer layer and nitinol wires are not shown.

FIGS. 11A-11C are perspective views showing the steering tube 300 attached to the gimbal 222 and coupler 224 including the nitinol wires 110a, 110b. The exploded view of FIG. 1B illustrates the different components of the assembly including the gimbal 222 and coupler 224. Note the angled tab 301 on the distal end 301b of the nitinol steering tube 300. During sheathing of the prosthesis (the coaptation sail), tab 301 is generally aligned with the axis of the steering tube; whereas upon unsheathing it flexes inwardly into the position shown. This is due to the spring property of the tab material.

The cross-sectional view of FIG. 11C illustrates the attachment of the coupler 224 to the steering tube 300 via pins 300a. Note that the gimbal is contained within, captured by, and extends through the proximal and distal portions of the coupler, 224a, 224b, respectively.

The purpose of the steering tube 300 is to position the coaptation sail 108 relative to the TV annulus. The multi-axis adjustability of this design, flexure in several planes, and rotation relative to the stent, collectively enable the position of the coaptation sail to be finely tuned to a patient's anatomy.

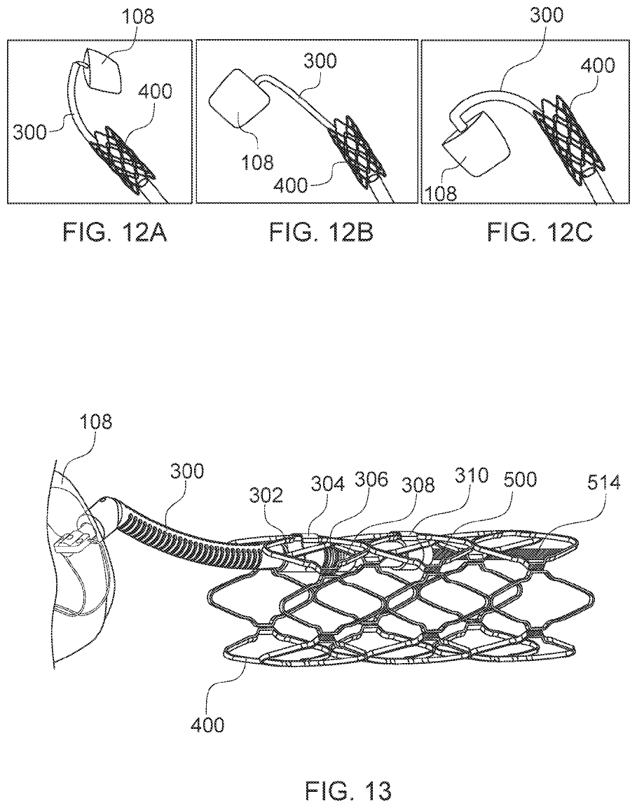

The schematic views of FIGS. 12A-12C illustrate several orientations of one steering system at various flexure amounts and rotations. Note that steering tube flexure is a result of the threaded insert (interacts with the tension rod subassembly) being rotated to increase the tension in the tensioning member which effectively shortens whichever side of the steering tube (where material is removed) to create curvature. The steering tube material is typically nitinol but other materials (PEEK, stainless steel, etc.) are also suitable.

One configuration of the steering system subassembly is shown assembled in FIG. 13. The illustrations in FIG. 15 and FIG. 17 are cross-sectional views of the steering configuration of FIG. 13 and further include details of the tensioning rod subassembly coupled to the steering rod subassembly.

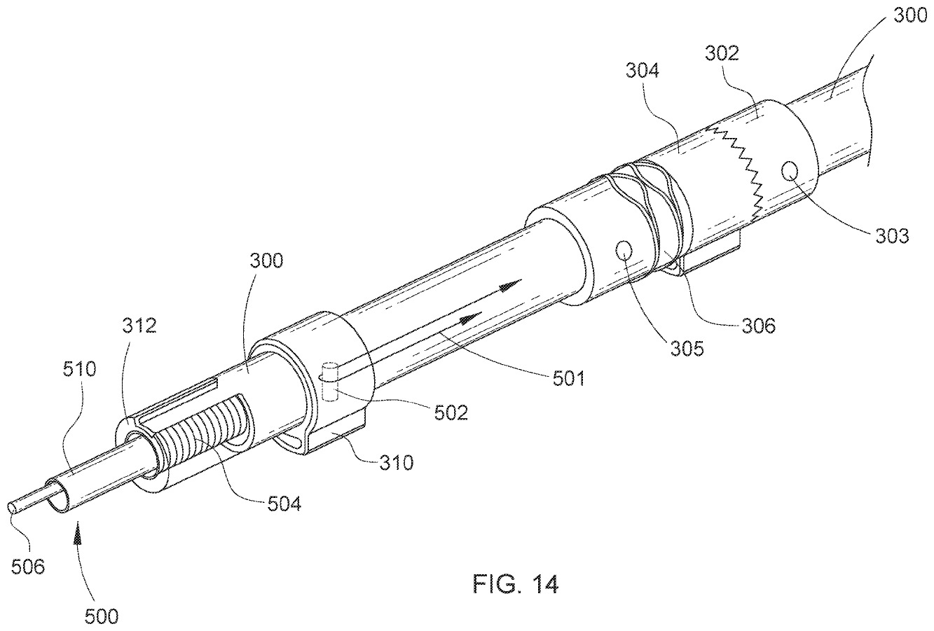

FIG. 14 is an upper perspective view showing the components comprising the steering system and tensioning rod subassemblies, here also illustrating how the tensioning member 501 (e.g., suture thread, fine cable or chain, medical wire, etc.) wraps around a suture pin 502 (i.e., an anchor pin). Note that as the threaded component 504 rotates to create tension, it imparts little to no moment to the tensioning member 501, and the axial load on the threaded components effectively locks it into position, as no counter torque is present for unthreading.

The individual components and respective functions of the multi-directional positioning and placement system notably includes a novel steering tube 300, as described above. It is to be understood that based upon the as-cut pattern (and additional cross-through pins), the flexure may occur in several different directions. This may be determined according to patient anatomy and control system requirements for a particular procedure. A serrated collar 302 is affixed to the steering tube 300 using a cross-through pin 303, which provides serrations on the inboard/proximal end and thereby locks the steering tube rotation angle to a serrated stent collar 304 having serrations that interdigitate and mate with those of the serrated collar. This provides flexure direction via tensioning member routing below cross through pin (see esp. FIGS. 15 & 17). The serrated stent collar 304 attaches the steering tube 300 to the stent 400 while allowing steering tube rotation relative to the stent.

A compression spring 306 provides spring force to engage the serrations of serrated collar 302 to serrated distal (first) stent collar 304 while allowing manual rotation of the steering tube relative to the stent. In embodiments, the compression spring 306 may be internal (not shown) to the steering tube to provide the locking spring force.

Although use of a serrated collar is shown, alternative embodiments include a tapered collet, which provides higher angular rotation resolution, or a cross pin and grooved collar arrangement, etc. An alternative embodiment (also not shown here) may enable compression of the spring, rotation of the steering tube, and locking the rotation angle via the delivery system handle controls.

A ring collar 308 attaches to the steering tube also using a cross-through pin 305 and counteracts the spring force of the compression spring 306. A proximal (second) stent collar 310 attaches to the steering tube and allows rotation and translation of the steering tube relative to the stent.

A tensioner rod subassembly 500 provides a secure connection between the prosthesis and the delivery system handle to deliver rotational (torque) forces to the steering tube through the tension rod and thereby to adjust tension for steering tube flexure.

The tensioning rod subassembly illustrated in FIGS. 15A-15C is shown in three views: an isometric assembly view, an exploded perspective view, and a cross-sectional perspective view. The individual components and respective functions shown include a suture pin 502 (FIG. 15B) affixed to the threaded tensioner 504 and connected to the tensioning member 501. The threaded tensioner 504 component, when threaded in or out of the threaded insert 312, adjusts the tension in the tensioning member to provide flexure to the steering tube 300. Balled wire 506 is combined with the threaded tensioner 504 using an balled expansion 506a at the distal end of the balled wire, which is captured in and between shaped recesses 504a and 510a in the proximal end of the threaded tensioner and the distal end 510a of the tension interlock 510 encircled by a tension collar 508, to provide an interface that locks into position to provide a torque-able assembly while allowing disconnection when the tension interlock 510 is translated away from the threaded tensioner 504. The balled wire is connected at its proximal end to the release button 604 in the delivery system. The tensioner collar 508 is affixed to cover the proximal end 504a of the threaded tensioner 504 and the distal end 510a of the tension interlock 510. The threaded tension interlock 510 is affixed to the tension tube 514 and the torsion tube 514.

A radiopaque band 512 is affixed over the tension tube 514 and provides fluoroscopic imaging aid in evaluating the relative position of the threaded tensioner 504 inside the threaded insert 312. The tension tube 514 is affixed to tension interlock 510 and connected to an operator control knob in the delivery system (not shown in these views).

The interlock assembly's individual components are shown side-by-side in FIG. 16. Although this illustration shows the components set apart, the final assembly is co-axial in nature. Here also the tension interlock 510 has been rotated 180-degrees to illustrate the end feature that captures the balled end 506a of the balled wire 506. As noted, the expanded spherical end of the balled wire 506 fits into the pocket at the end of the threaded tensioner 504 and when the tension interlock 510 is positioned over the round end, along with the tensioner collar 508 over all components at the interface, the balled wire is completely captured. Both the threaded tensioner 504 and tension interlock 510 have a “D” shaped end that when together are contained within the tensioner collar 508. This interlock assembly provides an interface that locks into position to provide a torque-able assembly while allowing disconnection when the tension interlock 510 is translated away from the threaded tensioner 504.

Although this configuration described a “captured balled wire in a pocket”, this is not limiting: alternatives include an L-shaped wire end that fits into either an L-shaped pocket in either side of the D-shaped ends or a slot with a hole in the end for the L-shaped wire end. Each alternative would require a tensioner collar to constrain the joint until disconnection is desired.

A cross-sectional view of the steering system and tensioning rod subassemblies is shown in FIGS. 17 and 19. Note the routing of the tensioning member (i.e., suture, wire, cable, chain, etc.) in this configuration is from a distal end 516, under medial point 518, to proximal return 520, and back to 516 along the same route. The typical assembly method entails routing the tensioning member inside a protective lubricious tube (i.e., FEP, PTFE, etc.) that loops around pin 520 and ties off at 516 while passing under cross-through pins 518. The protective lubricious tubing (not shown) prevents damage to the tensioning member from the inside edges of the steering tube 300 during flexure or natural prosthesis movement in the clinical setting. Additional cross through pins may be distributed throughout the length of the steering tube to create additional pivot points which, when combined with various laser-cut patterns, provide multi-directional steering tube flexure.

The tensioning member path over/under or from one side to the other of each cross through pin may vary according to the flexure desired. Additional guides may be placed on the cross through pins controlling the path of the tensioning member. Additional tensioning members may connect to these cross-through pins to enable various amounts of force applied to different sections of the steering tube through the use of co-axial or non-co-axial threaded inserts and tensioning rod configurations (not shown).

The attachment of the threaded insert to the steering tube, wherein the steering tube has “T” shaped features that interlock with the threaded insert, provides securement without fasteners or adhesives.

As can be seen in FIGS. 17-18, the stent 400 is configured for attachment to the steering tube. Serrated stent collar 304 and stent collar 310 each include two pins passing through the collars into aligned holes 402, 404 in the stent (see FIG. 18) for attachment of the steering tube to the stent. A stent strut gap between holes allows the one-piece stent collars to be securely captured on the stent strut.

A single co-axial tensioning rod subassembly is shown in FIG. 19. As may be surmised thus far, the purpose of the tensioning rod subassembly is to adjust the flexure of the steering tube and once positioned, to fixate the amount of tension, then disconnect from the prosthesis upon completion of the implantation procedure. The tensioning rod subassembly is detailed in each of FIGS. 15A-FIG. 17, and FIGS. 19-20. The connection between the steering system subassembly and the tensioning rod subassembly is achieved through the threaded insert 312, attached to the steering tube and threaded to threadably connected with a threaded tensioner 504.

The proximal end of the delivery system, as it relates to the tensioning rod subassembly is operatively coupled to a conventional endoscopic control handle, having a tension knob, a release knob, a guide wire lumen and luer lock, multiple dials to either rotate and/or flex the steering tube, levers for locking or unlocking the coaptation device's position, and other actuation mechanisms to control the multi-directional positioning and placement of the coaptation device.

The main components of the control handle for the delivery system are illustrated in FIGS. 21-22C. Referring to each and all of these views, the system at its proximal end includes a prosthesis delivery system handle 600, which provides multiple functions during the preparation and implantation of tricuspid valve prosthesis and provides a controlled implantation allowing retrieval, if needed. The control handle includes a tension knob 602, a release knob 604, and a guide wire lumen and luer lock 606. A flush-port 608 allows flushing the system with heparinized saline to remove all air from the inner catheter and prosthesis. A sheath dial 610 is operatively connected to a delivery sheath and retracts the outer sheath upon rotation to slowly expose the coaptation prosthesis. A heparinized saline drip line 612 promotes non-coagulation of the adjustment mechanisms during prosthesis delivery. A stent release button 614 prevents accidental prosthesis release until pushed by preventing the outer sheath from fully retracting. A cylindrical passage 620 in the control handle routes the tension tub 514 to an operative connection to a tension dial knob 602 which adjusts the amount of tension applied to the prosthesis upon rotation, and the release knob button 604 disconnects the delivery system from the prosthesis. Cylindrical passage 622 accepts the cardiac guide wire, which passes through the handle and out the luer lock 606 at the proximal end of the handle.

In FIGS. 22A-22C, note should be made of the delivery system handle 600, tension knob 602, release knob 604, and guide wire lumen and luer lock 606. The cross-sectional view (FIG. 22B) illustrates how each component is structurally and operationally related and how the compression spring inside the tension knob 602 that applies spring force to keep the interlock assembly connected. A side set screw 605 is included for safety to ensure the two components remain connected. When ready to disconnect, the side set screw is loosened allowing the tension knob 602 to be retracted for disconnection from the prosthesis.

Extended view (FIG. 22C) illustrates white ring visual indicators 607 that provide an applied tension reference point. Additional delivery system handle configurations (not shown) include multiple dials to either rotate and/or flex the steering tube, levers for locking or unlocking the coaptation device's position, and other control and actuation mechanisms for multi-directional movements of the coaptation device to ensure very precise positioning and placement in the TV.

The implantation procedure using the coaptation device and its delivery system resembles other transcatheter procedures using fluoroscopic and echogenic visualization and includes the following steps:

First, the femoral vein is accessed and an anatomical and TVR assessment is performed. Next, the coaptation prosthesis is prepared and sheathed and system preparation is verified. After that, a heparinized saline pressure bag is connected to the side stopcock of the delivery system handle and the bag pressure is set accordingly to ensure slight flow through sheath tip. The prosthesis is loaded in the delivery system. The physician/operator next advances the coaptation prosthesis and its control mechanism over the guide wire through the access site into the right atrium, using image guidance. The physician/operator will then observe the radiopaque nosecone and outer sheath tip marker using fluoroscopy.

To deploy the coaptation prosthesis, the delivery handle is pinned to a surface, and the sheath dial is rotated (CW), such that the tip of the outer sheath retracts and gradually exposes the sail into the right atrium, during which an outer sheath slides through the introducer sheath. The sheath dial rotation is stopped when the coaptation sail and the steering tube are entirely unsheathed. At this point, an assessment is made as to the coaptation sail position in relation to the TV annulus and its interaction with the native leaflets.

The sail is repositioned as needed for optimal results, either by: (1) advancing, retracting or rotating the entire prosthetic system; (2) further rotating the sheath dial (CW) to expose more of the prosthesis; or (3) rotating the tension knob (CCW) to flex the distal portion of the stent, with due caution taken to ensure this this action is taken only the stent is exposed.

Changes in regurgitation and valve function may then be assessed with ultrasound imaging (ICE, TTE).

Prosthesis Deployment: To deploy the prosthesis, the physician/operator carefully rotates the sheath dial 610 (CW) until it stops to expose the stent while maintaining the position of the distal edge of stent in the IVC. Note that the stent remains constrained in the sheath at its proximal end, and the stent is in apposition in the IVC during expansion.

Using a hex key provided in sterile prep materials, the operator carefully loosens the set screw 605 that connects the tension knob 602 to the release button 604. To disconnect the delivery system firm the stent, the operator provides a gentle push/pull action with the tension knob and release button—the tension knob providing the pull, and the release button providing a push. The result is carefully observed and disconnection is verified by carefully pulling the tension knob with the release button away from the handle 600.

To completely release the stent in the IVC a push and hold of the release button on the delivery system handle is employed, followed by rotation of the sheath dial (CW) until the stent is fully expanded in the IVC. (Note that the order of some steps may be reversed if appropriate under the circumstances.) The operator must then verify that the delivery system is fully detached from the prosthesis by gently advancing/retracting the delivery system.

The sheath dial is then rotated (CCW) to advance the sheath to the nosecone.

Finally, the guide wire is removed prior to the stent release, and it is removed from the now implanted and positioned prosthetic system.

The foregoing description is directed to preferred embodiments of the invention, including the best mode for carrying out the invention currently contemplated by the inventors. However, these do not exhaust or even begin to exhaust possible alternative embodiments, whether those are found in substantially equivalent alternative structures or substantially equivalent alternative operations, or both. The embodiments are, instead, described and presented for illustrative purposes, while it will be understood by those with skill in the art that this is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features, alternative order of method steps or the like. Therefore, the above description and illustrations should not be construed as limiting the scope of the invention, which is defined by the appended claims.

What is claimed as invention is:

1. A tricuspid valve prosthesis for treating tricuspid valve regurgitation (TVR), comprising:
a stent configured for implantation in the inferior vena cava (IVC) proximate the juncture with the right atrium (RA);
a coupler and gimbal assembly tethered to said stent; and
a coaptation sail having a wire frame connected to said coupler and gimbal assembly and enclosed within a material cover, said coaptation sail configured for percutaneous delivery to the RA for deployment within the tricuspid valve (TV) annulus;
wherein when said stent is implanted in said IVC and said tethered coaptation sail is deployed within the TV annulus, said coaptation sail material absorbs and retains blood such that blood coagulates within said coaptation sail, and over a plurality of cardiac cycles said coaptation sail is shaped by pressure differentials between the RA and right ventricle, blood flow, and physical engagement with native leaflets of the TV to form a three-dimensional shape that provides coaptation surfaces for the native leaflets and reduces the size of coaptation gaps, thereby reducing TVR.

2. The tricuspid valve prosthesis of claim 1, wherein said tether is a wire.

3. The tricuspid valve prosthesis of claim 1, wherein said stent is fabricated from nitinol.

4. The tricuspid valve prosthesis of claim 1, wherein said wire frame is fabricated from nitinol wire.

5. The tricuspid valve prosthesis of claim 4, wherein said wire frame is configured with curvature to match native leaflets before implantation.

6. The tricuspid valve prosthesis of claim 1, wherein said material cover is porous.

7. The tricuspid valve prosthesis of claim 1, wherein said material cover is non-porous.

8. The tricuspid valve prosthesis of claim 1, wherein said coupler and gimbal assembly includes a coupler having a proximal portion tethered to said stent, a distal portion pivotally connected to said proximal portion, said distal portion having a cylindrical through passage, a gimbal having a head captured between said proximal and distal portions of said coupler and a cylindrical shaft inserted through said through passage, said shaft having a distal end configured to attach to said wire frame of said coaptation sail.

9. The tricuspid valve prosthesis of claim 8, wherein said head of said gimbal and said distal portion of said coupler are configured to enable said gimbal to rotate about its longitudinal axis and to pivot in relation to said coupler.

10. The tricuspid valve prosthesis of claim 9, wherein said coupler and gimbal assembly is configured to provide multi-axial rotation of the coaptation sail relative to said coupler within the TV annulus

11. The tricuspid valve prosthesis of claim 10, wherein said coaptation sail has generally flat sides, a top side, and a bottom edge, and is configured to auto-rotate in relation to said coupler when deployed such that said top is parallel to the TV annulus to maximize coaptation with the native leaflets.

12. The tricuspid valve prosthesis of claim 10, wherein said cylindrical shaft of said gimbal has attachment structure at a distal end for connecting said wire frame.

13. The tricuspid valve prosthesis of claim 12, wherein said attachment structure includes a male threaded portion on said distal end of said cylindrical shaft and a wing nut threadably attached to said male threaded portion, wherein the wires of said wire frame are captured by and secured to said cylindrical shaft by said wing nut.

14. The tricuspid valve prosthesis of claim 9, wherein said gimbal includes a hemispherical ball under said head which engages a surface on said distal portion of said coupler and thereby facilitates a swiveling motion in relation to said distal portion of said coupler.

15. The tricuspid valve prosthesis of claim 1, further including a torsion spring disposed between said proximal and distal portions of said coupler so as to urge said proximal and distal portions into an angled orientation in relation to one another when deployed from a delivery sheath.
